(19) United States
(12) Patent Application Publication
(10) Pub. No.: US 2016/0008624 A1
(43) Pub. Date: Jan. 14, 2016

(54) DEVICES AND METHODS FOR TREATMENT INSIDE A BODY CAVITY

(71) Applicant: Jessica D Grossman, Tiburon, CA (US)

(72) Inventor: Jessica D Grossman, Tiburon, CA (US)

(21) Appl. No.: 14/794,913

(22) Filed: Jul. 9, 2015

Related U.S. Application Data
(60) Provisional application No. 62/023,295, filed on Jul. 11, 2014.

(51) Int. Cl. A61N 5/06

(52) U.S. Cl. CPC A61N 5/0603; A61N 5/0624; A61N 2005/061; A61N 2005/0611; A61N 2005/0626; A61N 2005/0651; A61N 2005/0654; A61N 2005/0661; A61N 2005/067

(57) ABSTRACT

A treatment device and method for transmitting UVC light energy within a patient's body cavity, the device having a distal end having a generally cylindrical shaped housing configured for insertion into the body cavity, a UVC light coupled to the housing configured to shine light radially outward from the housing; and a power source couple to the UVC light.

### CROSS-REFERENCE TO RELATED APPLICATIONS

The present application claims priority to U.S. Provisional Patent Application No. 62/023,295, filed Jul. 11, 2014, incorporated by reference herein in its entirety.

### FIELD

The present invention is generally related to treatment inside a body cavity, and more particularly, to the medical treatment of diseases of the female and male perineum and urinary/reproductive tract.

### BACKGROUND

Currently many disorders of the male and female urinary and reproductive tract (including the outer skin and mucous membranes covering these areas) are treated with drugs that are used either locally or systemically.

Common disorders include sexually transmitted infections (Gonorrhea, Chlamydia, HPV, Herpes, HIV, Syphilis, etc.), skin conditions (such as various forms of dermatitis, psoriasis, vitiligo, hidrandenitis supprativa, tineas, chron's disease, lichen sclerosis) as well as other infections that are not necessarily transmitted via sexual contact (streptococcal and staphylococcal infections, candida, yeast, bacterial vaginosis). Almost all of these diseases are currently treated by medications.

One such common disorder of the female reproductive tract is bacterial vaginosis, which is a multifactorial clinical syndrome with complex etiologies. The vaginal microbiome forms a mutually beneficial relation with its host and in turn has a major impact on both health and disease. In asymptomatic healthy women, several kinds of vaginal microbiota exist. The majority is dominated by lactobacillus and a diverse array of anaerobic organisms which play a protective role in preventing colonization by pathogens such as those responsible for bacterial vaginosis, yeast infections, sexually transmitted infections and urinary tract infections. The disturbance of this natural bacterial community results in what is commonly known as bacterial vaginosis, a highly prevalent vaginal disorder in reproductive age women with diagnostic and treatment options that are still ineffective. Bacterial vaginosis is characterized by a disruption of the “normal” vaginal flora. The prevalence of this disorder can be as high as 30% of women and has been shown to be an independent risk factor for STI's, HIV and pelvic inflammatory disease. It is commonly diagnosed by Amsel criteria or Nugent's score.

Common treatment is by drug ingestion or localized drug application (intravaginal) of metronidazole but 15-30% of women are symptomatic again within 30 to 90 days of antibiotic treatment. Metronidazole is a drug that is largely used for anaerobic infections. It is metabolized by the liver and other drugs may alter its concentration in the plasma. Metabolism of alcohol may also be affected resulting in intolerance in some patients. Different mechanisms of resistance are emerging and include reduced drug activation, inactivation by alternate pathways, prevention of drug entry and altered DNA repair.

Because of the increasing emergence of antibiotic resistance and the large proportion of patients who either will not take medication or are non-compliant with medication, there is a need for alternate forms of treatment of common pathogens. Indeed, many infectious disorders caused by pathogens are currently becoming multi-drug resistant due to the worldwide excessive use of antibiotics. Recently a new mutation has emerged that makes some bacteria resistant to almost all antibiotics is the USA in patients with urinary tract infections (MMWR 2010). In addition, Gonorrhea, a common sexually transmitted pathogen is becoming multidrug resistant and new treatment modalities are needed.

Thus there is a need for alternative treatment strategies and regimes to these common infectious disorders.

### SUMMARY

It is an object of the invention to provide a device and method for medical treatment of diseases of the female and male perineum and urinary/reproductive tract with UVC light, which as of yet not been commonly employed. The invention is a device that can be used to treat infections or other diseases that occur in both males and females in and around the perineal area (generally defined as the area between the pubic symphysis and the coccyx). This device supplies a method in which to treat or prevent infections in an individual user caused by microbes, bacteria, fungi, viruses and the like by applying electromagnetic radiation in the form of UV light in the range of UVC for a time, proximity and intensity sufficient to render said microbes inactive or incapable of reproduction.

Ultraviolet light is a form electromagnetic irradiation with a wavelength in the range of 100-400 nm. UV irradiation can be further subdivided into vacuum UV (100-200 nm), UVC (200-280 nm), UVB (280-315 nm) and UVA (315-400 nm). UVC light has long been recognized to be highly germicidal due to its ability to damage the genetic material of a cell. However its short wavelength renders it much less penetrant and thus less detrimental to human cells. Generally, UVC light permeates through the cell wall and into the cytoplasmic membrane of a microorganism causing molecular rearrangement of the microorganisms DNA. The extremely short wavelength of the UVC light makes penetration into human cells limited.

UVC light could also be combined with photo-modulation via a co-delivered drug treatment. There are also scenarios where photoluminescence with drug co-treatment could be utilized to deliver more or less UVC effects. In addition, the UVC treatment could be paired with or rapidly followed with probiotic treatment in order to restore healthy microbiobes after treatment and destruction of pathogenic bacteria.

The mechanism of inactivation of microorganisms via UVC is by causing chemical damage in the structure of DNA chains, specifically by cyclobutane pyrimidine dimers. A major advantage of UV treatment is that is relatively inexpensive, fast acting and in general safe. It can also be used on microbes that are multi-drug resistant. UVC is also known to be less damaging to human tissue than UVB (Gupta et al. 2013 Advances in wound care) light. However, devices to supply treatment to the genitorurinary tract with UVC has as of yet not been commonly employed.

### BRIEF DESCRIPTION OF THE DRAWINGS

FIG. 1 shows one embodiment of a probe device for use in a body cavity having a wand-like configuration.

FIG. 2 shows one embodiment of a probe device for use in a body cavity having a tampon-like configuration.

FIG. 3 shows one embodiment of a probe device for use in a body cavity having a plug-like configuration.

FIG. 4 shows one embodiment of a probe device for use in a body cavity having an insertable/implantable ring-like configuration.

FIG. 5 shows one embodiment of a probe device for use in a body cavity having an insertable/implantable diaphragm-like configuration.

FIG. 6 shows one embodiment of a probe device for use in a body cavity having a female condom configuration.

FIG. 7 shows one embodiment of a probe device for use in a body cavity having a male condom configuration.

FIGS. 8A and 8B show embodiments of a treatment device having a catheter-like configuration.

FIGS. 9A and 9B show one embodiment of a treatment device for treating the external skin and genitalia.

### DETAILED DESCRIPTION

Embodiments of the invention will now be described with reference to the figures, wherein like numerals reflect like elements throughout. The terminology used in the description presented herein is not intended to be interpreted in any limited or restrictive way, simply because it is being utilized in conjunction with detailed description of certain specific embodiments of the invention. Furthermore, embodiments of the invention may include several novel features, no single one of which is solely responsible for its desirable attributes or which is essential to practicing the invention described herein.

Ultraviolet (UV) light is a form electromagnetic irradiation with a wavelength in the range of 100-400 nm. UV irradiation can be further subdivided into vacuum UV (100-200 nm), UVC (200-280 nm), UVB (280-315 nm) and UVA (315-400 nm). UVC light has a short wavelength which renders it much less detrimental to human cells than UVB and UVC light. UVC light permeates through the cell wall and into the cytoplasmic membrane of a microorganism causing molecular rearrangement of the microorganisms DNA, making it highly germicidal due to its ability to damage the genetic material of the pathogenic cell. The extremely short wavelength of the UVC light makes penetration into human cells limited, so it is safer to use than UVA or UVB light for the treatment disclosed herein.

The mechanism of inactivation of microorganisms via UVC is by causing chemical damage in the structure of DNA chains, specifically by cyclobutane pyrimidine dimers. A major advantage of UVC treatment is that is relatively inexpensive, fast acting and in general safe. It can also be used on microbes that are multi-drug resistant. UVC is also known to be less damaging to human tissue than UVB light (see, Gupta et al. 2013 Advances in wound care).

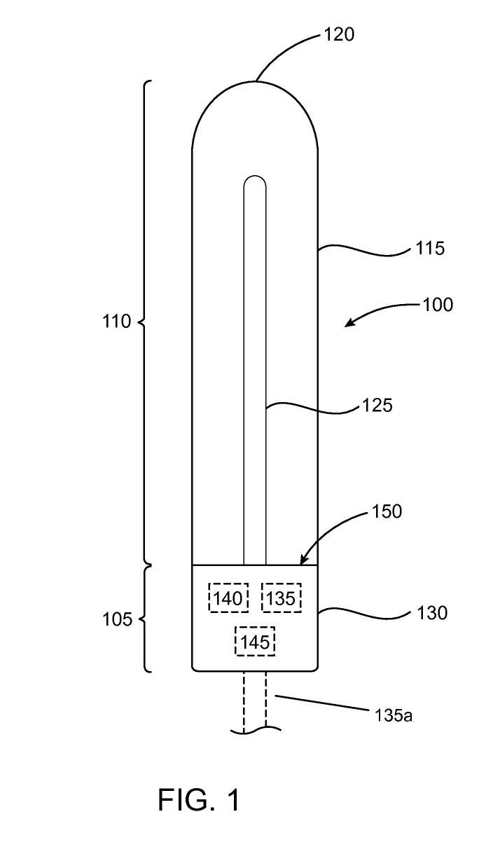

FIG. 1 shows one embodiment of the invention utilizing a UVC probe device 100 or insertion tube that would be inserted into a body cavity. The UVC probe is capable of shining ultraviolet (UV) light at wavelengths between 200-280 nm, and in some embodiments, at 254 nm. Portions of the probe device may utilize a transparent housing made of a UVC transmissive material such as polyurethane or Teflon or a quartz tube. The UV transmissive device and probe may be either rigid or flexible with the UV light source enclosed in a protective casing. In some embodiments, the UV light source is disposable, the power source is disposable, or a sheath covering the light source could be disposable. The UV light source would have a controller attached to it to control intensity, duration, wavelength, etc. of the light source. This could consist of a smart programmable interface, which could control all aspects of treatment, time, pulse, etc.

UVC light could be provided by an excimer laser, a laser diode, dye laser, metal halide lamp, UV LED, cold cathode fluorescent lamp (or other fluorescent light source), flashlamp, microwave generated UV plasma, and the like.

Other embodiments of the invention may include a flat or rounded window that would be made of a transparent material for the emission of the UV light. The other functional side may be shielded from UV light, and thus could be held by a user, the UV light being emitted is against the skin, tissue or mucous membrane to be treated, but not exposed to the hand holding the treatment device. An example of transparent material is Teflon or any other material that has a low refractive index.

In another embodiment of the invention there could be a small catheter like probe with UV emissive light sources (such as UV LED's) placed along the catheter with transparent flexible housing around these sections. This would enable treatment of the urethra or bladder, the oropharynx or a fistula or tract in the perineal area specifically. In another embodiment, UVC LED's could be embedded in a ring shaped device that could be inserted into the vagina and worn around the cervix similar to current drug eluting contraceptive devices such as the “Nuvaring”. Diaphragm like devices (or other barrier methods) of contraception (male or female condoms) could also have UVC emitting light sources embedded within them for treatment.

Differentiation is due to nothing disclosed in the prior art related to a probe specifically transmitting UVC light based therapy. This probe could also be modified for treatment of the perineal external surface such as the surface area of the vulva or placed into a flexible catheter based design for treatment of the urethra and bladder, the oropharynx or a fistula or tract in the perineal area. The device could be dually capable of insertion into a perineal orifice (vagina, urethra, fistula or anus) or for external surface treatment of the surrounding skin.

In some embodiments, the UVC treatment could be applied to the potentially affected orifice in the perineum either before or after sexual activity. UVC light therapy or prevention may also be used for reduction in biofilm growth. Desired treatment could be to remove or prevent bacterial or fungal colonization in the time frame of less than 1 minute to no more than 30 minutes. Treatment would result in disinfection (99% or 4 log kill) vs. sterilization. Irradiance ranges should be in the range of 0.1 mW/cm2 to 1 W/cm2, with radiant exposure should be in the range of 1 mJ/cm to 1 J/cm. Treatment would be by direct exposure of the infected or potentially infected area (such as the vaginal or oropharyngeal mucosae or epithelial cells or the skin or the urethra) with the UVC emissive device for a defined time period with a pre-programmed wavelength, irradiance and emission.

In other embodiments, the UVC probed device may include: sensors, controllers, on/off switches, timers, moving shields on a transparent window, intravaginal ring with UVC LED's, tampon shaped insertable with “string” to remove, optical fibers.

FIG. 1 shows one embodiment of a probe device 100 for use in a body cavity. Device 100 includes a proximal end 105 and a generally cylindrical distal end 110 configured for insertion into a desired body cavity. While the body cavity generally disclosed herein is a vagina, other body cavities may also take advantage of the device, such as oral, nasal, rectal, urinary, ear, and uterus cavities or other body cavities.

The distal end 110 includes a housing 115 with a domed tip 120 and one or more UVC light sources 125 radiating therapeutic UVC light at 200-280 nm radially outward from the housing 115. In the embodiment shown, the UVC light source 125 is a bulb position within a transparent housing 115. In other embodiments, one or more UVC light sources may be positioned on the inside or an outside surface of the housing 115. Optionally, a transparent cover may be positioned over the distal end 110. The cover may be disposable, i.e., single use, or may be a reusable cover.

The proximal end 105 may include a handle 130 coupled to the housing 115. The handle may include a power source 135, a controller 140 and one or more switches or controls 145. A shielding material 150 may be incorporated into the handle so that the one or more UVC light sources do not shine toward the proximal end at the user. The power source 135 is coupled to the one or more UVC light sources 125 and should be sized to provide enough power for the desired treatment profile. In various embodiments, the power source 135 may be one or more batteries, or may have a cord and plug 135a for attachment to an electrical outlet, such as in a house or a 12 v car outlet. The controller 140 is configured to control the UVC light source treatment profile. In some embodiments, the controller may be an external controller that is directly connected to the probe device, or may be wirelessly coupled to the probe device, for example by: Wi-Fi, Bluetooth, Radio Frequency Identification (RFID), Near Field Communications (NFC) or other suitable communication means. In some embodiments, the switch 145 is used to turn the system on and off. In other embodiments, one or more switches or dials 145 may be coupled to the controller to program the controller for the desired treatment profile.

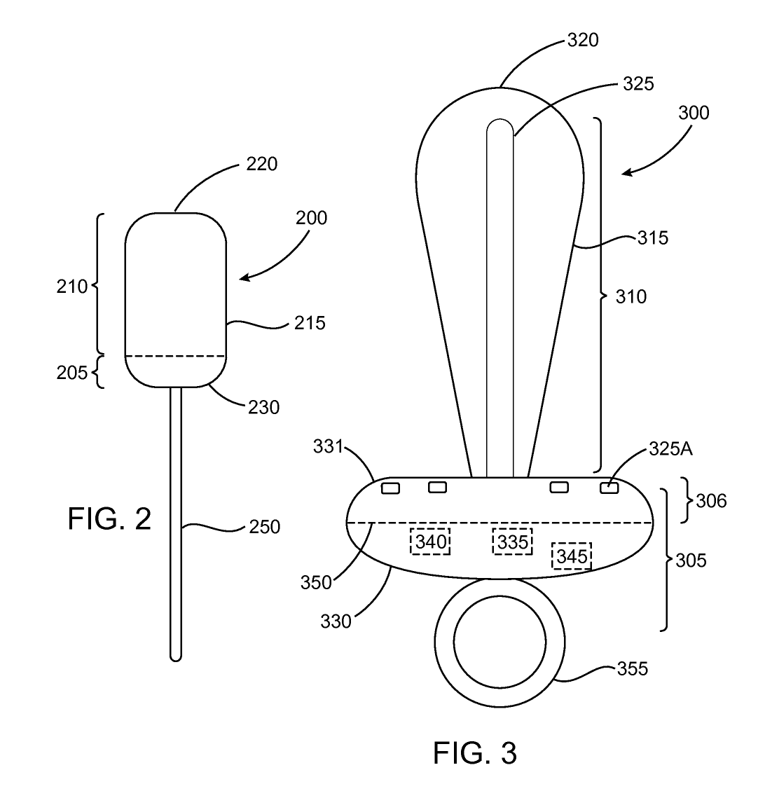

FIG. 2 shows another embodiment of an insertable probe device 200 for use in a body cavity. The probe device 200 is similar to probe device 100 except it has a tampon-like configuration for insertion into a vagina or anus. The probe device 200 includes a proximal end 205 and a generally cylindrical distal end 210 shaped for insertion into a desired body cavity.

The probe device 200 has a housing 215 with a domed tip 220. The proximal end 205 includes a portion 230 sized for a power source, a controller and one or more switches. A cord or string 250 may extend from the proximal end 205 and be coupled to the switches or controller. In some embodiments, the probe device 200 may be designed to be left in place, so the user may continue normal activities while being treated, with one treatment or multiple treatments over time. After the treatment is completed, the user pulls on the cord or string to remove 250. The probe device 200 may be a disposable device (one time use) or may be cleaned and reused. If reusable, the power source may be replaceable or may use rechargeable batteries.

FIG. 3 shows another embodiment of a probe device 300 that may be used to treat both a body cavity along with the external portions surrounding the body cavity. This embodiment has a plug-like shape having a proximal end 305 and a generally cylindrical distal end 310 shaped for insertion into a desired body cavity. The distal end 310 includes a housing 315 with a domed tip 320 and one or more UVC light sources 325 radiating therapeutic light at 200-280 nm radially outward from the housing 315. Similar to the other embodiments, the one or more UVC light sources may be within a transparent housing 315, positioned on an outside surface of the housing 315.

The proximal end 305 includes a disk shaped handle 330 coupled to the housing 315. The handle may include a power source 335, a controller 340 and one or more switches or controls 345. In the embodiment shown, the handle 330 also includes one or more UVC light sources 325a on a distal side 331 aimed toward the distal end. The one or more UVC light sources may be within the housing 315 transmitting through a transparent portion of the distal side 331, or may be positioned on an outside surface of the distal side 331. A shielding material 350 may be incorporated into the handle so that the one or more UVC light sources do not shine toward the proximal end at the user. The handle 330 may also include a ring or string 355 to position and/or remove the probe device 300 after treatment.

The power source 335 is coupled to the one or more UVC light sources 325 and should be sized to provide enough power for the desired treatment profile. In some embodiments, the power source 335 may be one or more batteries, rechargeable batteries, or may have a cord and plug for attachment to an electrical outlet, such as in a 120 v house outlet or a 12 v car outlet. The controller 340 is configured to control the UVC light source treatment profile. In some embodiments, the switch 345 is used to turn the system on and off. In other embodiments, the switch 345 may be an assortment of dials couple to the controller to program the controller for the desired treatment profile.

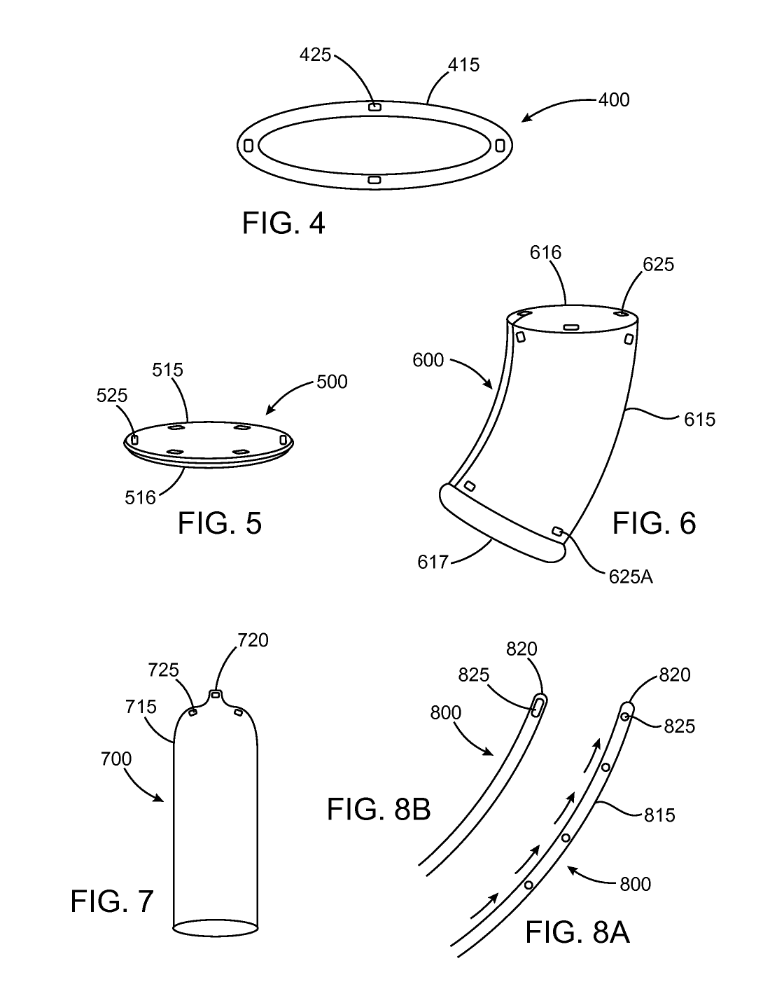

FIG. 4 shows one embodiment of an insertable/implantable treatment device 400 having a ring shaped housing 415 made of a material that is flexible enough to be inserted and positioned within a body cavity, such as a vagina. The housing 415 includes one or more UVC light sources 425 radiating therapeutic light at 200-280 nm radially outward from the housing 415. The one or more UVC light sources 425 may be positioned within a transparent housing 415, or positioned on an outside surface of the housing 415. The housing 415 may include a power source, a controller, and one or more switches or controls. The housing 415 may also include a shielding material so that the one or more UVC light sources shine in the desired direction.

FIG. 5 shows another embodiment of an insertable/implantable treatment device 500, similar to treatment device 400, having a housing 515 that is similar to a diaphragm, made of a soft latex or silicone material with a rim 516. A spring may be molded into the rim to create a seal against the walls of the vagina. The housing 515 includes one or more UVC light sources 525 radiating therapeutic light at 200-280 nm outward from the housing 515. The one or more UVC light sources 525 may be position within a transparent housing 515, or positioned on an outside surface of the housing 515. The housing 515 may include a power source, a controller, and one or more switches or controls. The housing 515 may also include a shielding material so that the one or more UVC light sources shine in the desired direction.

FIG. 6 shows another embodiment of insertable/implantable treatment device 600 having a housing 615 in the shape of a female condom. In some embodiments, the device 600 is a thin, soft, loose-fitting sheath or housing 615 with a flexible ring at each end. The inner ring 616 at the closed end of the sheath is used to insert the condom inside the vagina and to hold it in place during intercourse. The rolled outer ring 617 at the open end of the sheath remains outside the vagina and covers part of the external genitalia. One or more UVC light sources 625 may be positioned on an outside and/or inner surface of the housing 615. One or more UVC light sources 625a may also be positioned near a proximal end. The housing 615 may include a power source, a controller, and one or more switches or controls. The housing 615 may also include a shielding material so that the one or more UVC light sources shine in the desired direction.

FIG. 7 shows another embodiment of a treatment device 700 having a housing 715 in the shape of a male condom. The housing 715 may be made from latex, but some are made from other materials such as polyurethane, polyisoprene, or lamb intestine. One or more UVC light sources 725 may be positioned on an outside surface of the housing 715. The housing 715 may include a power source, a controller, and one or more switches or controls. The housing 715 may also include a shielding material so that the one or more UVC light sources shine in the desired direction. The housing 715 may also include a ring at the open end of the sheath, similar to rolled outer ring 617, and may further include one or more UVC light sources aimed toward the distal end, similar to UVC light sources 325a.

FIGS. 8A and 8B show embodiments of a treatment device 800 having a housing 815 in the shape of a urinary catheter configured for insertion into a patient's bladder via the urethra or oropharynx via the mouth or a fistula tract via its distal end. The housing 815 may be made of a latex, polyurethane, or silicone tube. In FIG. 8A, a plurality of UVC light sources 825 may be positioned along the length of the housing 815. In FIG. 8B, a single UVC light source 825 may be positioned at the distal or tip 820 of the housing 815. The UVC light source(s) may be coupled to a power source, a controller, and one or more switches or controls at a proximal end.

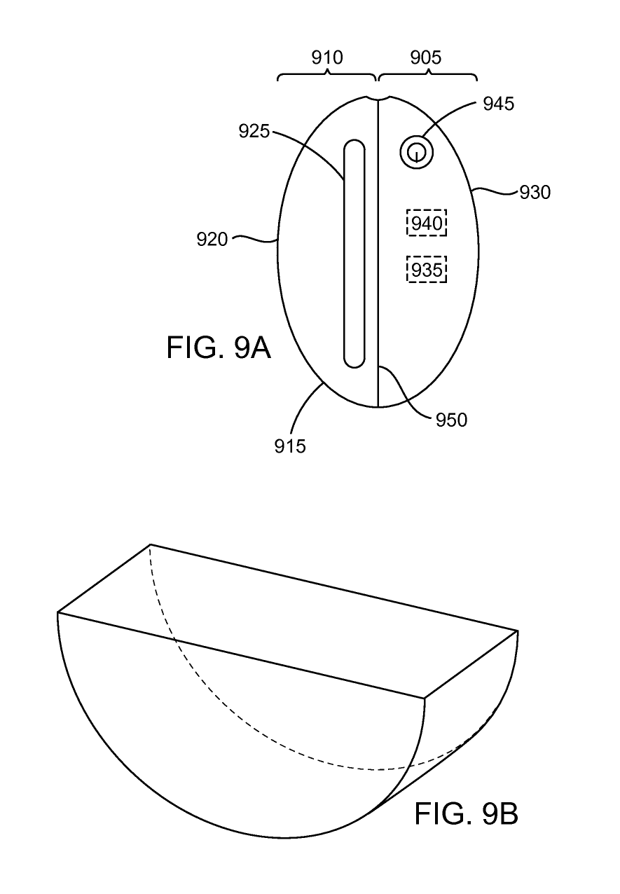

FIGS. 9A and 9B show another embodiment of a treatment device 900 for treating the external perineal skin or genitalia. The treatment device 900 includes a housing 915 having a hand-held shape with proximal portion 905 and distal portion 910. The distal portion 910 includes a housing 915, shown with a domed shape 920 and one or more UVC light sources 925 radiating therapeutic light at 200-280 nm radially outward from the housing 915. The light sources may be a one or more bulbs, a 3D array, LEDs, or other suitable UVC light sources. In the embodiment shown, the UVC light source 925 is a bulb position within a transparent housing 915. In other embodiments, the one or more UVC light sources may be positioned on an outside surface of the housing 915. Optionally, a transparent cover may be positioned over the distal end 910. The cover may be disposable, i.e., single use, or may be a reusable cover.

The proximal portion 905 may include a handle 930 coupled to the housing 915. The handle may include a power source 935, a controller 940 and one or more switches or controls 945. The power source 935 is coupled to the one or more UVC light sources 925 and should be sized to provide enough power for the desired treatment profile. In some embodiments, the power source 935 may be one or more batteries, or may have a cord and plug for attachment to an electrical outlet, such as in a house or a 12 v car outlet. The controller 940 is configured to control the UVC light source treatment profile. In some embodiments, the switch 945 is used to turn the system on and off. In other embodiments, the switch 945 may be an assortment of dials couple to the controller to program the controller for the desired treatment profile. A shielding material 950 may be incorporated into the handle so that the one or more UVC light sources do not shine toward the proximal end at the user.

For the UVC light therapy to perform properly, the UVC light source must shine on the desired tissue or treatment area. One way to shine the light around the body cavity and manually move the treatment device to the desired tissue or treatment area. If the treatment device is positioned within the body cavity while the patient is moving around, such as the tampon embodiment, the treatment device may also move around and expose light to different areas of the desired tissue or treatment area naturally.

In some embodiments the treatment device may include a touch sensor or a pressure sensor to control the on/off status of the UVC light source in such a way that the light source can only be turned on when the treatment device touches the body cavity tissue. This helps to improve the safety of the device by limiting inadvertent light exposure.

In some embodiments the treatment device may include a temperature sensor to monitor the temperature of the body cavity tissue. The obtained temperature signal can be utilized for controlling the UVC light energy that is delivered to the tissue. When the measured tissue temperature reaches dangerous or undesirable levels, a warning signal can be sent to the operator to shut down the treatment device.

It is believed that the construction, operation and advantages of this invention will be apparent to those skilled in the art. It is to be understood that the present disclosure is illustrative only and that changes, variations, substitutions, modifications and equivalents will be readily apparent to one skilled in the art and that such may be made without departing from the spirit of the invention as defined by the following claims.

It is intended that the specification and examples be considered as exemplary only, with a true scope and spirit of the invention being indicated by the following claims. In addition, where this application has listed the steps of a method or procedure in a specific order, it may be possible, or even expedient in certain circumstances, to change the order in which some steps are performed, and it is intended that the particular steps of the method or procedure claims set forth herebelow not be construed as being order-specific unless such order specificity is expressly stated in the claim.

The invention claimed is:

1. A treatment device for transmitting UVC light energy within a patient's body cavity comprising:
a probe having a proximal and distal end, the proximal end having a handle and the distal end having a generally cylindrical shaped housing coupled to the handle, the cylindrical shaped housing being configured for insertion into the body cavity;
one or more UVC light sources radiating light between 200-280 nm, UVC light being coupled to the housing configured to radiate the UVC light radially outward from the housing; and
a power source couple to the one or more UVC light sources.

2. The treatment device of claim 1, wherein the housing is a transparent housing made of a UVC transmissive material and the UVC light is positioned within the transparent housing

3. The treatment device of claim 1, wherein the UVC light is positioned on a surface of the housing.

4. The treatment device of claim 1, wherein an outer surface of the housing is part of the UVC light.

5. The treatment device of claim 1, wherein the UVC light is selected from the group consisting of: an excimer laser, a laser diode, dye laser, metal halide lamp, UV LED, cold cathode fluorescent lamp (or other fluorescent light source), flashlamp, microwave generated UV plasma, and the like.

6. The treatment device of claim 1, wherein the housing is flexible.

7. The treatment device of claim 1, wherein the housing is rigid.

8. The treatment device of claim 1, further comprising a controller coupled to the one or more UVC light sources, the controller being configured to control the light emissions from the one or more UVC light sources for a desired treatment.

9. The treatment device of claim 8, wherein the controller may control the intensity, duration, or wavelength of the one or more UVC light sources.

10. The treatment device of claim 8, wherein the controller is positioned within the handle.

11. The treatment device of claim 1, wherein the power source is positioned within the handle.

12. The treatment device of claim 11, wherein the power source is one or more replaceable or rechargeable batteries.

13. The treatment device of claim 1, further comprising one or more switches on the handle coupled to the power source.

14. The treatment device of claim 1, further comprising a transparent cover configured to fit over the distal end.

15. The treatment device of claim 11, wherein the cover may be disposable or reusable.

16. The treatment device of claim 1, further comprising a shielding material configured to direct the radiation from the one or more UVC light sources in a treatment direction.

17. The treatment device of claim 1, wherein the handle also includes one or more UVC light sources on a distal side aimed toward the distal end.

18. The treatment device of claim 1, further comprising a ring or string coupled to the handle configured to position and/or remove the probe device.

19. A tampon treatment device for transmitting UVC light energy within a vagina comprising:
a probe having a generally cylindrical tampon shaped housing sized to fit completely within the vagina during a treatment, the housing having a proximal and a distal end configured to be insertable into the vagina;
one or more UVC light sources radiating light between 200-280 nm being positioned on or within the housing and being configured to radiate the UVC light radially outward from the housing; and
a power source couple to the one or more UVC light sources positioned within the housing.

20. The treatment device of claim 19, further comprising a controller positioned within the housing coupled to the one or more UVC light sources, the controller being configured to control the light emissions from the one or more UVC light sources for a desired treatment.

21. The treatment device of claim 20, wherein the controller may control the intensity, duration, or wavelength of the one or more UVC light sources.

22. The treatment device of claim 19, further comprising a ring or string coupled to the probe configured to position and/or remove the probe.

23. A treatment device for transmitting UVC light energy within a patient's body cavity comprising:
a ring shaped housing sized to fit within the body cavity during a treatment;
one or more UVC light sources radiating light between 200-280 nm being positioned on or within the housing and being configured to radiate the UVC light radially outward from the housing; and
a power source couple to the one or more UVC light sources positioned within the housing.

24. The treatment device of claim 23, further comprising a controller coupled to the one or more UVC light sources, the controller being configured to control the light emissions from the one or more UVC light sources for a desired treatment.

25. The treatment device of claim 24, wherein the controller may control the intensity, duration, or wavelength of the one or more UVC light sources.

26. A treatment device for transmitting UVC light energy within a patient's vagina comprising:
a diaphragm shaped device sized to fit within the vagina, the device being made of a flexible material coupled to an outer ring, the outer ring being configured to seal against the walls of the vagina;
one or more UVC light sources radiating light between 200-280 nm being positioned on or within the device and being configured to radiate the UVC light outward from the device; and
a power source couple to the one or more UVC light sources positioned within the device.

27. The treatment device of claim 26, further comprising a controller coupled to the one or more UVC light sources, the controller being configured to control the light emissions from the one or more UVC light sources for a desired treatment.

28. The treatment device of claim 27, wherein the controller may control the intensity, duration, or wavelength of the one or more UVC light sources.

29. A condom device for transmitting UVC light energy comprising:
a condom shaped device being made of a flexible material;
one or more UVC light sources radiating light between 200-280 nm being positioned on or within the device and being configured to radiate the UVC light in a desired direction from the device; and
a power source couple to the one or more UVC light sources.

30. The treatment device of claim 29, further comprising a controller coupled to the one or more UVC light sources, the controller being configured to control the light emissions from the one or more UVC light sources for a desired treatment.

31. The treatment device of claim 30, wherein the controller may control the intensity, duration, or wavelength of the one or more UVC light sources.

32. The treatment device of claim 30, wherein the condom shaped device is a female condom sized to fit within the vagina.

33. The treatment device of claim 30, wherein the condom shaped device is a male condom sized to fit on a penis and configured to be insertable into the vagina, wherein the one or more UVC light sources radiate outwardly from the device.

34. A urinary treatment device for transmitting UVC light energy within a patient's urinary tract comprising:
a probe having a generally cylindrical housing configured to be insertable into the patient's urinary tract;
one or more UVC light sources radiating light between 200-280 nm being positioned on or within the housing and being configured to radiate the UVC light radially outward from the housing; and
a power source couple to the one or more UVC light sources positioned within the housing.

35. The treatment device of claim 34, further comprising a controller coupled to the one or more UVC light sources, the controller being configured to control the light emissions from the one or more UVC light sources for a desired treatment.

36. The treatment device of claim 35, wherein the controller may control the intensity, duration, or wavelength of the one or more UVC light sources.
